(19) United States
(12) Patent Application Publication
(10) Pub. No.: US 2006/0148067 A1
(43) Pub. Date: Jul. 6, 2006

(54) Bio-bonding detection apparatus using ultrasonic waves and method thereof

(76) Inventors: Tae-sik Park, Suwon-si (KR); Young-il Kim, Suwon-si (KR); Jung-ho Kang, Suwon-si (KR); Nam-su Park, Suwon-si (KR)

Correspondence Address: CANTOR COLBURN, LLP, 55 GRIFFIN ROAD SOUTH, BLOOMFIELD, CT, 06002, US

(21) Appl. No.: 11/282,233

(22) Filed: Nov. 17, 2005

(30) Foreign Application Priority Data
Jan. 6, 2005 (KR) 10-2005-0001286

(51) Int. Cl. C12M 1/34

(52) U.S. Cl. 435/287.2

(57) ABSTRACT

An apparatus for detecting bio-bonding using ultrasonic waves and a method thereof are disclosed. The apparatus includes a generator for generating ultrasonic wave, a transformer, disposed in proximity to the generator, the transformer configured for vibration thereof by the generated ultrasonic wave. A probe biomolecule is immobilized on the transformer, and a metal plate is disposed in proximity to the transformer. An analyzer is configured for measuring the capacitance between the transformer and the metal plate. The bio-bonding detection apparatus conveniently determines whether bio-bonding is generated by detecting variation in capacitance or variation in a characteristic of the ultrasonic wave.

This application claims priority to Korean Patent Application No. 2005-1286, filed on Jan. 6, 2005, and all the benefits accruing therefrom under 35 U.S.C. §119, the disclosure of which is incorporated herein by reference.

### BACKGROUND OF THE INVENTION

1. Field of the Invention

The present invention relates in general to an apparatus for detecting bio-bonding and a method thereof. More particularly, the invention relates to an apparatus and method for detecting bio-bonding using ultrasonic waves

2. Description of the Related Art

A biochip is a biological microchip. It can be used to analyze, for example, gene expression, distribution pattern and/or mutation. A biochip is manufactured by arranging hundreds to hundreds of thousands of biomolecules. The biomolecules on the chip can be, for example, nucleic acids (i.e., DNA or RNA), enzymes, or antibodies. The substrate can be formed, for example of glass, silicon, nylon or the like. The biochip can be a biosensor comprising a biomaterial and a physical, chemical or optical transformer. Specific examples of a biochip include a nucleic acid micro-array comprising a nucleic acid; a protein chip comprising a protein such as an enzyme, antibody or antigen; a cell chip comprising a cell; or a neuron chip comprising a nerve cell.

Recently, there have been many efforts to implement Lab-On-a-Chip (LOC) technology for use with biological molecules. The development of a DNA-LOC or a Protein-LOC is presently in progress. A DNA-LOC or a Protein-LOC is a chip capable of pre-processing, derivatizing, isolating, and analyzing biological samples, such as blood, urine, cell, sputum, or a food. The LOC is a chip integrated with the functions of an automatic analysis system, such as a valve for pretreatment, a reactor, an extractor, an isolating system, and a sensor technology, which are required to analyze a biochemical material.

A biochip includes a probe biomolecule immobilized on a surface of the biochip. When the probe biomolecule is mixed with a sample, it can search a sample for a component to which it can bind. A sample component to which the probe biomolecule can bind is referred to herein as a ligand. For example, when the probe biomolecule is a nucleic acid, the ligand can be a nucleic acid with a sequence with sufficient complementarity to the sequence of the probe nucleic acid to permit hybridization between the two nucleic acids. When a probe biomolecule is an enzyme, the ligand can be a molecule that binds to the enzyme. Other combinations of probe biomolecule and ligand will be known to the skilled practitioner. When a sample is placed on the biochip, components of the sample that are ligands of the probe biomolecule can bind with the probe biomolecule on the biochip. Information about the components of the sample can then be obtained by detecting and analyzing the probe biomolecule in the bound state.

Various technologies related to a biochip have been introduced, including technologies for immobilizing the probe biomolecule on the biochip, detecting a signal, and processing the signal information. Examples of signal detection technologies currently in use include optical bio-bonding detection methods, chemical bio-bonding detection methods, and mechanical bio-bonding detection methods.

FIGS. 1A to 1C illustrate a conventional optical bio-bonding detection method.

Conventional optical bio-bonding detection methods optically determine whether a probe biomolecule is bonded to components of a sample. In order to detect bio-bonding through the conventional optical bio-bonding detection method, sample components are labeled with a fluorescent material and the labeled sample is mixed with the probe biomolecule on the biochip such that ligands in the sample can bind with the probe biomolecule. The amount of binding is determined using a fluorescence detecting device. However, a disadvantage of this optical bio-bonding detecting method is that it requires pretreatment for adding the fluorescent material to components of the sample before the sample is mixed with the probe biomolecule. Therefore, the sample may be damaged or contaminated by the fluorescent material. Another disadvantage is that a high-cost optical reader is required for detecting the result, and analysis of the data from the optical reader is complicated. Furthermore, it is difficult to obtain digitized data from the optical bio-bonding detection method and very difficult to miniaturize an apparatus based on the optical bio-bonding detection method.

FIG. 1A shows probe biomolecules being mixed with sample components with a fluorescent label. FIG. 1B shows removal of sample components that are not bound to the probe biomolecules, for example, by washing. FIG. 1C shows irradiation of the bio-bonded probe biomolecules with light for determination of the amount of bio-bonding by analyzing the amount of fluorescence detected.

FIGS. 2A to 2C are views of several examples of conventional mechanical bio-bonding detection methods. In particular, FIG. 2A shows a mechanical bio-bonding detection method using a cantilever, FIG. 2B shows a mechanical bio-bonding detection method using a surface acoustic wave (SAW) biosensor, and FIG. 2C shows a mechanical bio-bonding detection method using a scanning probe microscope (SPM).

In FIG. 2A, a mechanical bio-bonding detection method using a cantilever is illustrated. Bio-bonding is detected by measuring and comparing intermolecular cohesion before and after the probe biomolecule is bound to a ligand. In order to measure the intermolecular cohesion, deflection of the cantilever beam must be accurately measured. Therefore, this mechanical bio-bonding detection method requires supplementary equipment such as a laser.

FIG. 2B illustrates a mechanical bio-bonding detection method using a SAW biosensor. A predetermined frequency signal is input to the SAW filter and a ligand from the sample binds to the probe biomolecule on the SAW filter. The bio-bonding is detected by observing a filtering variation of the SAW filter generated by binding of the ligand in the sample to the probe biomolecule.

The mechanical bio-bonding detection method using SPM illustrated in FIG. 2C also requires additional equipment such as a laser device and a photo diode.

FIG. 3 is a schematic diagram illustrating another conventional chemical bio-bonding detection method in which the detection of binding of probe biomolecules is based on detection of a chemical variation. A predetermined chemical material is placed on an electrode where the probe biomolecule and the sample are mixed, and an electrochemical reaction of the predetermined chemical material is observed. By measuring the electrochemical reaction level of the predetermined chemical material, bio-bonding is detected. However, electrochemical detection is comparatively less sensitive than optical bio-bonding detection methods.

FIGS. 4A to 4C are views of an electrical bio-bonding detection method using electrical variations to detect binding. FIGS. 4A and 4B illustrate an electrical bio-bonding detection method using a trench type capacitance element, while FIG. 4C illustrates an electrical bio-bonding detection method using a plane type capacitance element.

In FIGS. 4A to 4C, a capacitance element is used in the electrical bio-bonding detection method, wherein a variation of the capacitance value is observed for detecting bio-bonding. However, it is very difficult to miniaturize the capacitance element to implement in a biochip. Since capacitance is proportional to cross sectional area of the capacitance element and inversely proportional to the thickness of the capacitance element, it is very complicated to design a capacitance element to have a wider cross section that is suitable for bio-processing. In order to use the trench type capacitance element shown in FIGS. 4A and 4B for detecting bio-bonding, the trench is formed so as to have a wider cross section and a thinner thickness. However, since the gap is very small, it is relatively inefficient for bio-processing.

In the bio-bonding detection method using a plane type capacitance element shown in FIG. 4C, a capacitor having a capacitance element made of very thin metal plate formed in the shape of a comb is used for bio-bonding detection. However, the sensitivity of bio-bonding detection using this type of capacitance element is comparatively low.

### SUMMARY OF THE INVENTION

Accordingly, the present general inventive concept has been made to solve the above-mentioned problems, and an aspect of the present general inventive concept is to provide an apparatus for detecting bio-bonding by generating ultrasonic waves at the bottom of a transformer to which biomolecules are immobilized and measuring vibrational variation of the transformer resulting from the bio-bonding, and a method thereof.

Disclosed herein is a bio-bonding detection apparatus. In one embodiment, the apparatus includes a generator for generating an ultrasonic wave and a transformer, disposed in proximity to the generator, the transformer configured for vibration thereof by the generated ultrasonic wave. A probe biomolecule is immobilized on the transformer, and a metal plate is disposed in proximity to the transformer. An analyzer is configured for measuring the capacitance between the transformer and the metal plate.

The generator may be any one of an ultrasonic wave oscillator using a semiconductor, a magnetostriction oscillator and a crystal oscillator.

The transformer may be any one of a membrane, a vibrator that is vibrated by the ultrasonic wave, and a sonic filter for eliminating high frequency band noises.

The analyzer may be one of a CV converter converting the capacitance to a voltage, and a CI converter converting the capacitance to a current.

In another embodiment, the apparatus includes a generator for generating an ultrasonic wave, and a transformer, disposed in proximity to the generator, the transformer configured for vibration thereof by the generated ultrasonic wave. A probe biomolecule is immobilized on the transformer, and a metal plate is disposed in proximity to the transformer. A receiver is disposed on the metal plate, the receiver configured for analyzing a variation of a selected characteristic of the ultrasonic wave.

The generator may be one of an ultrasonic wave oscillator using a semiconductor, a magnetostriction oscillator and a crystal oscillator.

The transformer may be any one of a membrane, a vibrator that is vibrated by the ultrasonic wave, and a sonic filter for eliminating high frequency band noises.

The receiver can analyze a characteristic of the ultrasonic wave selected from frequency, amplitude, time delay, energy, phase variation or any combination of the foregoing.

Additionally disclosed herein are bio-bonding detection methods. In one embodiment, the method includes passing an ultrasonic wave through a transformer to which a probe biomolecule is immobilized, measuring a first capacitance value between the transformer and a metal plate disposed in proximity to the transformer, mixing the probe biomolecule and a sample, and determining the presence or absence of bio-bonding to the probe biomolecule by detecting a variation of the first capacitance value between the metal plate and the transformer from a second capacitance value measured after mixing with the sample.

The variation of the capacitance may be measured by using one of a CV converter converting the capacitance to a voltage, and a CI converter converting the capacitance to a current.

In another embodiment, the bio-bonding detection method includes passing an ultrasonic wave through a transformer to which a probe biomolecule is immobilized, analyzing one or more selected characteristics of the ultrasonic wave passed through the transformer, mixing the probe biomolecules and a sample, and re-analyzing the one or more selected characteristics of the ultrasonic wave passed through the transformer after the probe biomolecule is mixed with the sample.

The analyzed characteristic of the ultrasonic wave may be one of frequency, amplitude, phase, time delay, energy or any combination of the foregoing.

### BRIEF DESCRIPTION OF THE DRAWINGS

The above aspects and features of the present invention will be more apparent by describing certain embodiments of the present invention with reference to the accompanying drawings, in which:

FIGS. 1A to 1C illustrate conventional optical bio-bonding detection methods;

FIGS. 2A to 2C illustrate conventional mechanical bio-bonding detection methods;

FIG. 3 illustrates a conventional chemical bio-bonding detection method;

FIGS. 4A to 4C illustrate conventional electrical bio-bonding detection methods;

FIGS. 5A to 5B illustrate a bio-bonding detection apparatus using ultrasonic waves in accordance with an exemplary embodiment of the present invention;

FIGS. 6A to 6C illustrate an exemplary embodiment of a bio-bonding detection method using ultrasonic waves using the bio-bonding detection apparatus shown in FIG. 5A;

FIGS. 7A to 7C illustrates an exemplary embodiment of a bio-bonding detection method using ultrasonic waves using the bio-bonding detection apparatus shown in FIG. 5B; and

FIG. 8 is a flowchart of a bio-bonding detection method using ultrasonic waves in accordance with an exemplary embodiment of the present invention.

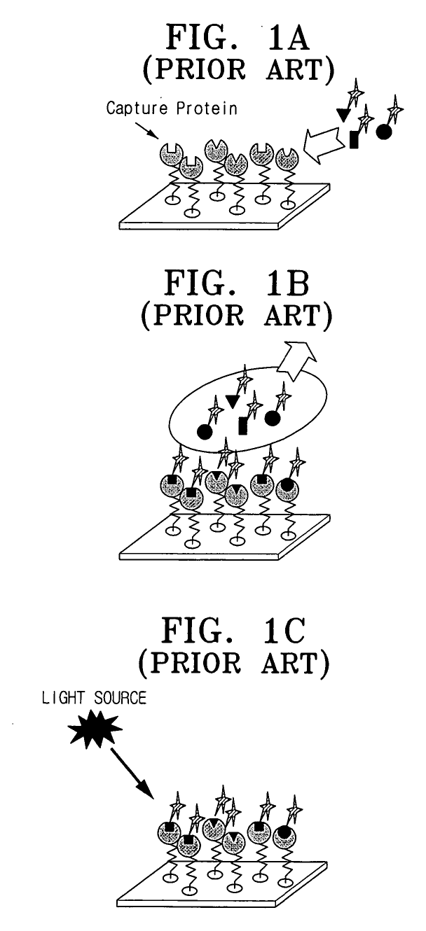

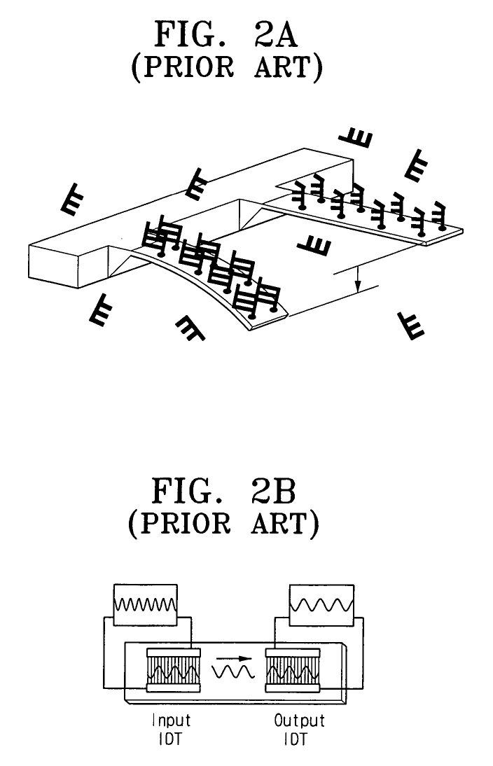

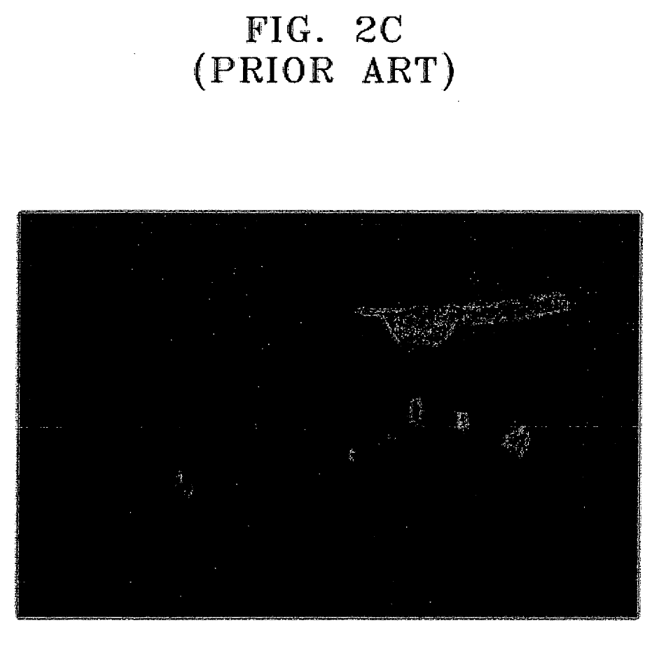

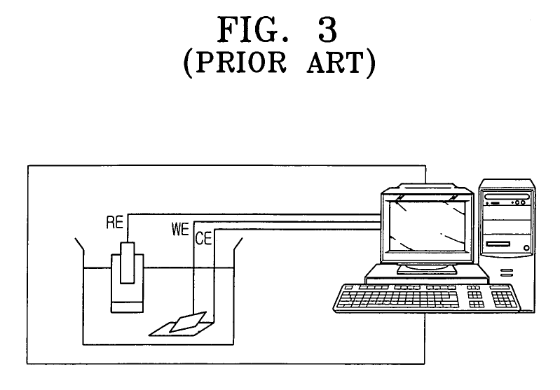

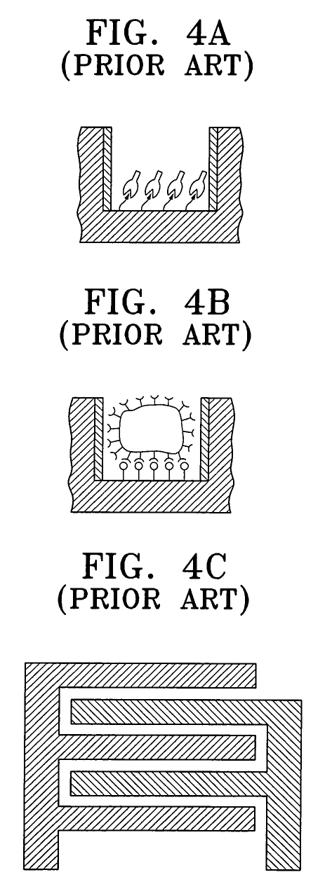

### DETAILED DESCRIPTION OF THE PREFERRED EMBODIMENTS

The invention is described more fully hereinafter with reference to the accompanying drawings, in which exemplary embodiments of the invention are shown. This invention may, however, be embodied in many different forms and should not be construed as limited to the exemplary embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity.

It will be understood that when an element or layer is referred to as being “on” or “connected to” another element or layer, the element or layer can be directly on or connected to another element or layer or intervening elements or layers. In contrast, when an element is referred to as being “directly on” or “directly connected to” another element or layer, there are no intervening elements or layers present. Like numbers refer to like elements throughout. As used herein, the term “and/or” includes any and all combinations of one or more of the associated listed items.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the invention are described herein with reference to schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

Also, well-known functions or constructions are not described in detail since they would obscure the invention in unnecessary detail.

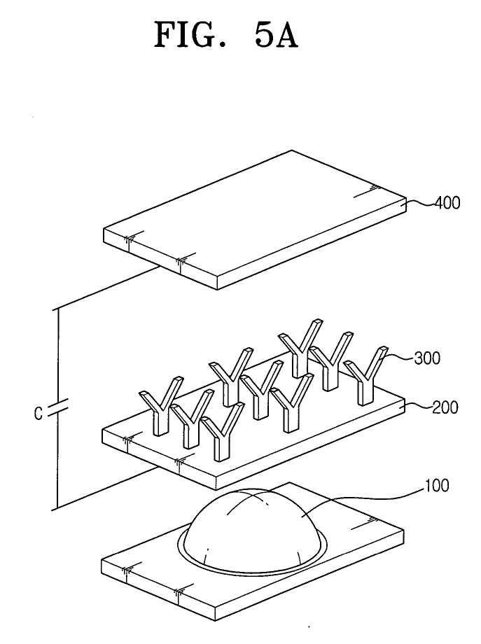

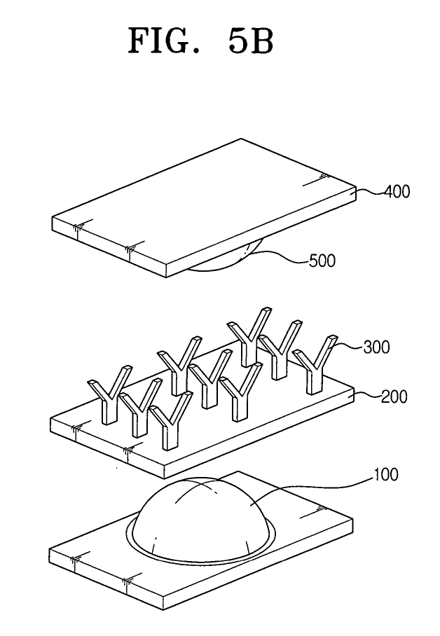

FIGS. 5A and 5B illustrate a bio-bonding detection apparatus using ultrasonic waves in accordance with an exemplary embodiment of the present invention. FIG. 5A shows a bio-bonding detection apparatus configured to detect changes in capacitance measurements before and after the bio-bonding to detect the bio-bonding, and FIG. 5B shows a bio-bonding detection apparatus using measurement of the ultrasonic wave signal characteristics before and after the bio-bonding to detect the bio-bonding.

In FIG. 5A, the bio-bonding detection apparatus includes a generator 100, a transformer 200, a probe biomolecule 300 and a metal plate 400.

The generator 100 generates ultrasonic waves having a frequency of about 200 KHz or higher. The generator 100 may be, for example, an ultrasonic wave oscillator using a semiconductor, a magnetostriction oscillator using magnetization of a metal, or a crystal oscillator that resonates by supplying a high frequency voltage to a crystal plate.

The transformer 200 is vibrated by the ultrasonic wave generated from the generator 100, and transforms the characteristics of the ultrasonic wave generated from the generator 100. The probe biomolecule 300 is immobilized on the transformer 200, and enables a sample to be searched for the presence of a ligand that binds probe biomolecule 300. The transformer 200 is disposed within a predetermined distance with respect to the generator 100. The transformer 200 may be a membrane or a vibrator, either of which are vibrated by the ultrasonic wave, or the transformer 200 can be a sonic filter eliminating noises of super high frequency.

When the probe biomolecule 300 immobilized on the transformer 200 is mixed with the sample and becomes bonded to a ligand in the sample, the vibration of transformer 200 (caused by the ultrasonic wave generated from generator 100) changes compared to its vibration when the probe biomolecule 300 is not bonded to a ligand in the sample, since the mass of the transformer 200 is changed by the ligand binding to the probe biomolecule 300.

The metal plate 400 is disposed at a predetermined distance with respect to the transformer 200. When a ligand in the sample is biologically bonded to the probe biomolecule 300 attached to the transformer 200, the vibrational motion of the transformer 200 is changed. Therefore, the distance between the transformer 200 and the metal plate 400 also changes, and the capacitance between the transformer 200 and the metal plate 400 also varies.

Accordingly, bio-bonding is detected by measuring the capacitance variation resulting from the change in distance between the metal plate 400 and the transformer 200. The capacitance variation between the metal plate 400 and the transformer 200 is analyzed by an analyzer (not shown). The analyzer may be, for example, a CV converter converting a capacitance to a voltage or a CI converter converting a capacitance to a current.

In the embodiment of FIG. 5B, the bio-bonding detection apparatus comprises a generator 100, a transformer 200, a probe biomolecule 300, a metal plate 400 and a receiver 500. As the configuration of the generator 100, the transformer 200, the probe biomolecule 300, and the metal plate 400 are identical to those shown in FIG. 5A, the detailed explanations thereof are omitted.

The receiver 500 receives the ultrasonic wave passed through the transformer 200 and analyzes the characteristics thereof. Such characteristics can include, for example amplitude, frequency, phase, energy, delay, or any combination thereof. As described previously, when the probe biomolecule 300 immobilized on the transformer 200 is bonded to a ligand in the sample, the mass of the transformer 200 increases by the mass of the ligand bonded to the probe biomolecule 300. Therefore, the characteristics of the ultrasonic wave passed through the transformer 200 changed as a result of the change in mass of transformer 200. Therefore, by comparing the characteristic of the ultrasonic wave passed through the transformer 200 before and after the probe biomolecule is mixed with the sample, the presence or absence of biological bonding between the probe biomolecule and a ligand in the sample can be detected.

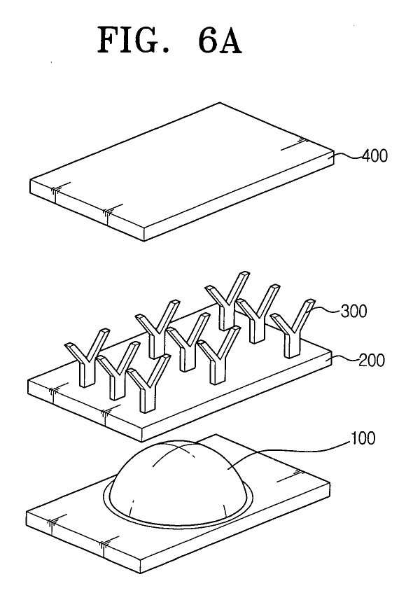

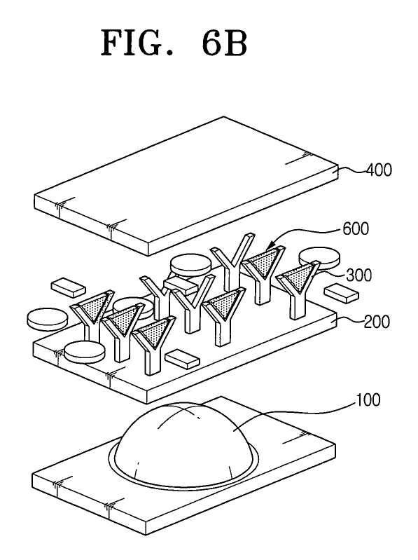

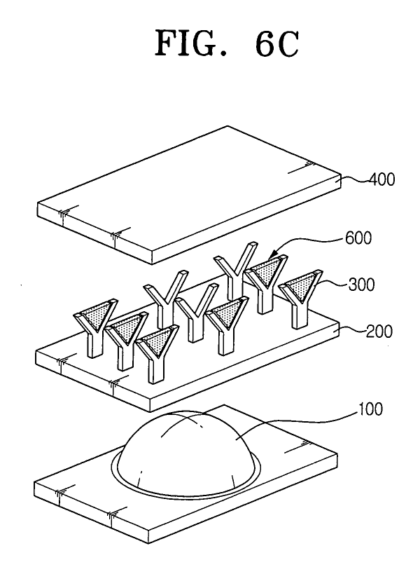

FIGS. 6A to 6C illustrate an exemplary embodiment of a bio-bonding detection method using ultrasonic waves using the bio-bonding detection apparatus shown in FIG. 5A. FIG. 6A shows the bio-bonding detection apparatus having probe biomolecule 300 immobilized on transformer 200 before any bio-bonding. FIG. 6B shows the bio-bonding detection apparatus with probe biomolecule 300 mixed with the sample. FIG. 6C shows the bio-bonding detection apparatus after removal of sample components that do not bind to probe biomolecule 300, for example by a washing step.

In FIGS. 6A to 6C, the probe biomolecule 300 is immobilized on the transformer 200 arranged between the generator 100 and the metal plate 400, and an ultrasonic wave is generated. After generating the ultrasonic wave, the capacitance between the transformer 200 and the metal plate 400 is measured before probe biomolecule 300 is mixed with the sample. Then, the sample is mixed with the probe biomolecule 300 and a sample ligand 600 is biologically bonded to the probe biomolecule 300, and unbound components of the sample are removed, for example by washing.

After removal of unbound sample components, an ultrasonic wave is generated and passed to the transformer 200, and the capacitance between the transformer 200 and the metal plate 400 is measured again. The determination of whether bio-bonding between the probe biomolecule 300 and the sample ligand 600 is present or absent can then be made by determining the difference between the before-bonding capacitance and the after-bonding capacitance.

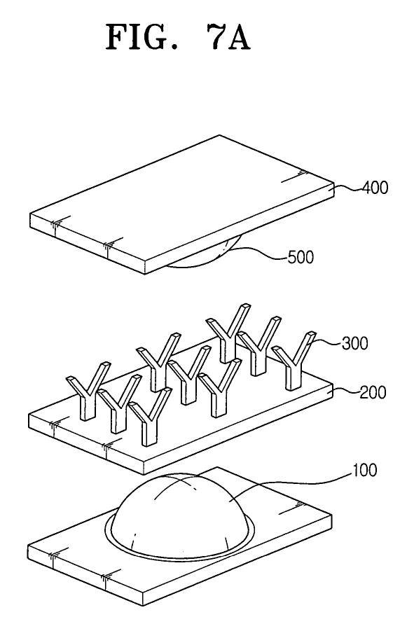

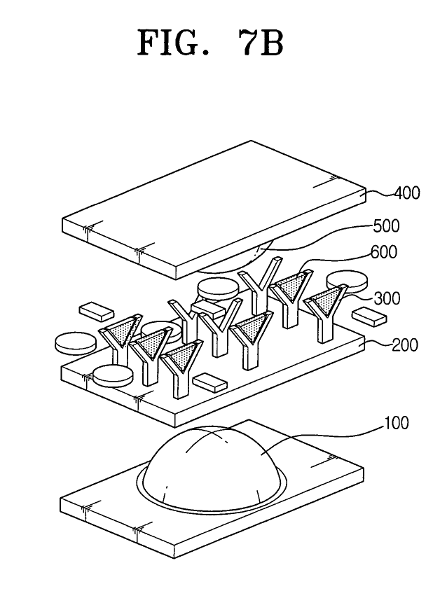

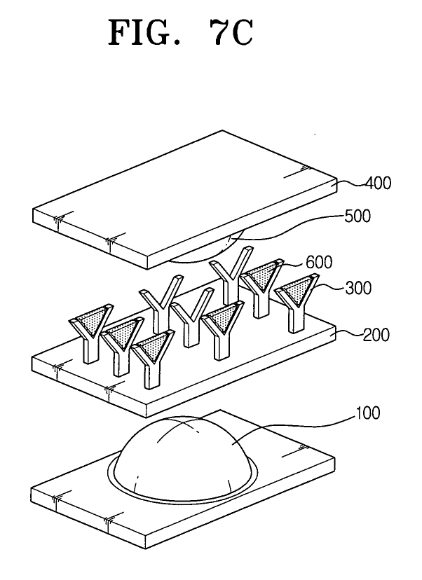

FIGS. 7A to 7C illustrate an exemplary embodiment of a bio-bonding detection method using ultrasonic waves using the bio-bonding detection apparatus shown in FIG. 5B. FIG. 7A shows a bio-bonding detection apparatus having probe biomolecule 300 immobilized on transformer 200 before probe molecule 300 is mixed with the sample. FIG. 7B shows the bio-bonding detection apparatus comprising a probe biomolecule 300 mixed with the sample. FIG. 7C shows the bio-bonding detection apparatus after removal of unbound components of the sample from the probe biomolecules 300.

In the method illustrated in FIGS. 7A to 7C, after immobilization of the probe biomolecule 300 on the transformer 200 arranged between the generator 100 and the metal plate 400 and before mixing with a sample, selected characteristics of the ultrasonic wave passed through the transformer 200 are analyzed to obtain a before-bonding value for the ultrasonic wave characteristic. The measured characteristic of the ultrasonic wave may include, for example frequency, amplitude, phase, energy, time delay, or any combination thereof.

The probe biomolecule 300 immobilized on the transformer 200 is mixed with the sample and can become biologically bonded to the sample ligand 600. Sample components that are not bound to the probe biomolecule 300 are removed by washing. After washing, the characteristics of the ultrasonic wave are analyzed again to obtain an after-bonding value. The determination of the presence or absence of bio-bonding between sample ligand 600 and probe biomolecule 300 is obtained by determining the difference between the before-bonding value and the after-bonding value of the characteristics of the ultrasonic wave.

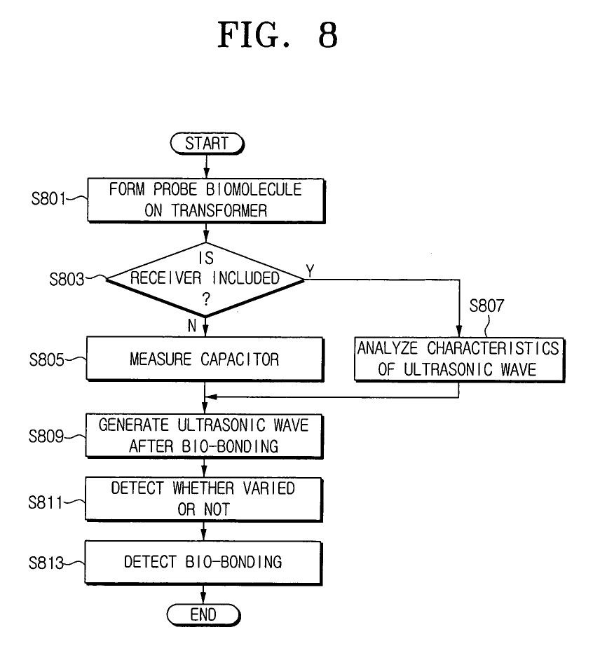

FIG. 8 is a flowchart of an exemplary embodiment of a bio-bonding detection method using ultrasonic waves in accordance with the present invention.

Referring to FIG. 8, probe biomolecule 300 is immobilized on the transformer 200 in operation S801. The transformer 200 is vibrated by the ultrasonic wave, thus changing the characteristics of the generated ultrasonic wave. The transformer 200 may be a membrane or a vibrator that is vibrated by the ultrasonic wave, or the transformer 200 may be a sonic filter eliminating super high frequency noises.

Then, operation S803 (decision block) determines the presence or absence of the receiver 500 in the apparatus used in performing the method. If receiver 500 is included in the apparatus and arranged on the metal plate 400, it receives and analyzes the characteristics of the ultrasonic wave passed through the transformer 200, as shown at operation S807. The characteristics may be, for example, frequency, amplitude, a phase, a time delay, an energy, or any combination thereof

On the other hand, if there is no receiver 500 included, the generated ultrasonic wave is passed through the transformer 200 to the metal plate 400 and the capacitance between the transformer 200 and the metal plate 400 is measured as shown in operation S805. In order to measure the capacitance, a CV converter converting a capacitance to a voltage, or a CI converter converting a capacitance to a current may be used. After measuring the before-bonding value for either capacitance (S805) or other characteristics of the ultrasonic wave (S807), the probe biomolecule 300 immobilized on the transformer 200 is mixed with the sample such that binding between probe biomolecule 300 and ligand 600 present in the sample occurs and components of the sample that are not bound to the probe biomolecule 300 can be removed by a washing step.

After the bio-bonding has occurred, an after-bonding value of the capacitance or the characteristic of the ultrasonic wave is measured in operation S809 and any difference in the before-bonding and after-bonding values is determined in operation S811. When the variation in capacitance is measured, the before-bonding capacitance is compared to the after-bonding capacitance in order to determine whether there is difference between the before-bonding capacitance and the after-bonding capacitance resulting from the fact that the mass of the transformer 200 becomes heavier when the probe biomolecules are biologically bonded to ligands. Such an increased mass of the transformer 200 changes the vibrational motion of the transformer 200, and the changed vibrational motion changes the capacitance.

Since the vibrational motion of the transformer 200 is changed as a result of the increased mass, the distance between the metal plate 400 and the transformer 200 is consequently changed. Accordingly, the capacitance therebetween is changed as well. The capacitance is proportional to the area (A) of two flat planes and to the permittivity ε of the medium between the two flat planes, and inversely proportional to the distance (d) between the two flat planes. The distance between the transformer 200 and the metal plate 400 is changed by vibrational variation of the transformer 200, thus permitting a determination of the presence or absence of bio-bonding by measuring the capacitance variation before and after mixing the probe biomolecules with the sample.

If characteristics of the ultrasonic wave are measured to determine presence or absence, and/or amount of bio-bonding occurring between the probe biomolecule 300 and a component of the sample, ligand 600, the characteristic(s) so analyzed may be frequency, amplitude, time delay, energy, phase or any combination thereof. As noted previously, the mass of the transformer 200 becomes heavier when the probe biomolecules are biologically bonded to the ligand component of the sample, resulting in changes in the vibrational motion of the transformer 200. If the ultrasonic wave is passed through the transformer 200 having increased mass, the characteristics of ultrasonic wave are changed.

The vibrational motion of the transformer 200 vibrated by the ultrasonic wave signal may be expressed by following equation:

$$f ∝ \sqrt{\frac{k}{m}} \qquad Eq . 1$$

wherein f denotes vibrational motion, k is an elastic coefficient, and m is mass of the transformer 200. Herein, the elastic coefficient k is proportional to the thickness of the transformer 200 and the probe biomolecule 300.

For example, if the density of the transformer 200 (e.g., made from silicon) is about 2.33 g/cm³, the dimensions of the transformer 200 are about 100 μm×100 μm×1 μm, the thickness of the transformer 200 is about 1 μm, the thickness of the probe biomolecule 300 is about 50 μm, and the elastic coefficient (k) is about 10 N/m, the variation (Δk) of elastic coefficient after bio-bonding is 0.5 [N/m]. The vibrational motion of the transformer 200 changes after the probe biomolecules are biologically bonded to ligands in the sample, and the variation of the vibrational motion changes both the capacitance between the metal plate 400 and the transformer 200, as well as the characteristics of the ultrasonic wave when the ultrasonic wave is passed through the transformer 200.

Bio-bonding is detected according to whether the variation of the capacitance or variation of the characteristic of the ultrasonic wave is detected or not in operation S813. That is, if the capacitance is varied or the characteristic of the ultrasonic wave is varied after the probe biomolecules 300 are biologically bonded to the ligands 600 from the sample, it is determined that bio-bonding occurred between the probe biomolecules 300 and the ligands 600.

As described above, the bio-bonding detection apparatus using ultrasonic waves according to the present invention can conveniently determine whether bio-bonding is generated or not by detecting a variation of the capacitance after the probe biomolecules are biologically bonded to the ligands in a sample or by detecting a variation of a characteristic of the ultrasonic wave after the ultrasonic wave passed through the transformer.

As will also be appreciated, the bio-bonding detection apparatus may be easily manufactured and can be embodied as a simply structured device.

The foregoing embodiment and advantages are merely exemplary and are not to be construed as limiting the present invention. The present teachings can be readily applied to other types of apparatuses. Also, the description of the embodiments of the present invention is intended to be illustrative, and not to limit the scope of the claims, and many alternatives, modifications, and variations will be apparent to those skilled in the art.

What is claimed is:

1. A bio-bonding detection apparatus, comprising:
a generator for generating an ultrasonic wave;
a transformer, disposed in proximity to the generator, the transformer configured for vibration thereof by the generated ultrasonic wave;
a probe biomolecule immobilized on the transformer;
a metal plate disposed in proximity to the transformer; and
an analyzer for measuring the capacitance between the transformer and the metal plate.

2. The bio-bonding detection apparatus of claim 1, wherein the generator is one of an ultrasonic wave oscillator using a semiconductor, a magnetostriction oscillator and a crystal oscillator.

3. The bio-bonding detection apparatus of claim 1, wherein the transformer is one of a membrane, a vibrator that is vibrated by the ultrasonic wave, and a sonic filter for eliminating high frequency band noises.

4. The bio-bonding detection apparatus of claim 1, wherein the analyzer is one of a CV converter converting the capacitance to a voltage, and a CI converter converting the capacitance to a current.

5. A bio-bonding detection apparatus, comprising:
a generator for generating an ultrasonic wave;
a transformer, disposed in proximity to the generator, the transformer configured for vibration thereof by the generated ultrasonic wave;
a probe biomolecule immobilized on the transformer;
a metal plate disposed in proximity to the transformer; and
a receiver disposed on the metal plate, the receiver configured for analyzing a variation of a selected characteristic of the ultrasonic wave.

6. The bio-bonding detection apparatus of claim 5, wherein the generator is one of an ultrasonic wave oscillator using a semiconductor, a magnetostriction oscillator and a crystal oscillator.

7. The bio-bonding detection apparatus of claim 5, wherein the transformer is one of a membrane, a vibrator that is vibrated by the ultrasonic wave, and a sonic filter for eliminating high frequency band noises.

8. The bio-bonding detection apparatus of claim 7, wherein the selected characteristic of the ultrasonic wave is one of frequency, amplitude, time delay, energy, phase variation, or any combination of the foregoing.

9. A bio-bonding detection method, the method comprising:
passing an ultrasonic wave through a transformer to which a probe biomolecule is immobilized;
measuring a first capacitance value between the transformer and a metal plate disposed in proximity to the transformer;
mixing the probe biomolecule and a sample; and
determining the presence or absence of bio-bonding to the probe biomolecule by detecting a variation of the first capacitance value between the metal plate and the transformer from a second capacitance value measured after mixing with the sample.

10. The method of claim 9, wherein the capacitance values are measured by using one of a CV converter converting the capacitance to a voltage, and a CI converter converting the capacitance to a current.

11. A bio-bonding detection method, the method comprising:
passing an ultrasonic wave through a transformer to which a probe biomolecule is immobilized;
analyzing one or more selected characteristics of the ultrasonic wave passed through the transformer;
mixing the probe biomolecules and a sample; and
re-analyzing the one or more selected characteristic of the ultrasonic wave passed through the transformer after the probe biomolecule is mixed with the sample.

12. The method of claim 11, wherein the analyzed characteristics of the ultrasonic wave is one of frequency, amplitude, phase, time delay, energy or any combination of the foregoing.

13. The method of claim 11, further comprising
determining the presence or absence of bio-bonding to the probe biomolecule by analyzing variation of the characteristic of the ultrasonic wave analyzed before and after mixing of the probe biomolecule with the sample.

14. The method of claim 11, further comprising
determining the extent of bio-bonding to the probe biomolecule by analyzing variation of the characteristic of the ultrasonic wave analyzed before and after mixing of the probe biomolecule with the sample.
